SEQUENCE LISTING (10 sequences)

SEQ ID NO: 1 - DNA, 81 bp, Mus musculus
atggcctcaccgttgacccgctttctgtcgctgaacctgctgctgctgggtgagtcgattatcctggggagtggagaagct

SEQ ID NO: 2 - DNA, 711 bp, Artificial Sequence
  Anti-ALK mouse scFv
caggtgcaattgcagcagtctggggctgaactggcaaaacctggggcctcagtgaagctgtcctgcaaggcttctggctacacctttactagctactggatgcactgggtaaaacagaggcctggacagggtctggaatggattggatacattaagcctagcagtggttatactaagtacaatcagaagttcaaggacaaggccacattgactgcagacaaatcctccagcacagcctacatgcagctgagcagcctgacatatgaggactctgcagtctattactgtgcaagagattactacggtagtagctcctggtttgcttactggggccaagggactctggtcactgtctccgcgggtggaggtggatcaggtggaggtggatctgacattgtgatgacccagtctcaaagattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcaacaatataacagctatccgtacatgtacacgttcggaggggggaccaagctcgag

SEQ ID NO: 3 - DNA, 186 bp, Mus musculus
tcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcct

SEQ ID NO: 4 - DNA, 81 bp, Mus musculus
ttttgggcactggtcgtggttgctggagtcctgttttgttatggcttgctagtgacagtggctctttgtgttatctggaca

SEQ ID NO: 5 - DNA, 123 bp, Mus musculus
aatagtagaaggaacagactccttcaaagtgactacatgaacatgactccccggaggcctgggctcactcgaaagccttaccagccctacgcccctgccagagactttgcagcgtaccgcccc

SEQ ID NO: 6 - DNA, 339 bp, Mus musculus
cgcgcgaaatttagccgcagcgcggaaaccgcggcgaacctgcaggatccgaaccagctgtataacgaactgaacctgggccgccgcgaagaatatgatgtgctggaaaaaaaacgcgcgcgcgatccggaaatgggcggcaaacagcagcgccgccgcaacccgcaggaaggcgtgtataacgcgctgcagaaagataaaatggcggaagcgtatagcgaaattggcaccaaaggcgaacgccgccgcggcaaaggccatgatggcctgtatcagggcctgagcaccgcgaccaaagatacctatgatgcgctgcatatgcagaccctggcgccgcgc

SEQ ID NO: 7 - DNA, 119 bp, Artificial Sequence
  Linker, cleavage sequence (Furin cleavage site, V5 epitope, Spacer, T2A)
gtgctaagcgtggtaagcctatccctaaccctctcctcggtctcgattctacgagcggaagcggagagggcagaggaagtctgctaacatgcggtgacgtcgaggagaatcctggacct

SEQ ID NO: 8 - DNA, 402 bp, Mus musculus
cacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctaa

SEQ ID NO: 9 - DNA, 23 bp, Artificial Sequence
  ALK gene cDNA-specific primer
tatacgaatgtcacatggacccc

SEQ ID NO: 10 - DNA, 23 bp, Artificial Sequence
  ALK gene cDNA-specific primer
tatatggaggaagtcttgccagc

(19) United States
(12) Patent Application Publication
(10) Pub. No.: US 2024/0066059 A1
(43) Pub. Date: Feb. 29, 2024

(54) METHOD FOR PRODUCING CHIMERIC ANTIGEN RECEPTOR-MACROPHAGES AND USE OF SAME CELLS

(71) Applicants: SEOUL NATIONAL UNIVERSITY R&DB FOUNDATION, Seoul (KR); KNU-INDUSTRY COOPERATION FOUNDATION, Gangwon-do (KR)

(72) Inventors: Byung Soo KIM, Seoul (KR); Mi Kyung KANG, Seoul (KR); Hee Ho PARK, Gangwon-do (KR)

(21) Appl. No.: 18/279,965

(22) PCT Filed: Mar. 3, 2022
(86) PCT No.: PCT/KR2022/003008

§ 371 Date: Sep. 1, 2023

(30) Foreign Application Priority Data
Mar. 3, 2021 (KR) 10-2021-0028253
Mar. 3, 2022 (KR) 10-2022-0027426

(51) Int. Cl. A61K 35/15; A61K 39/00; A61P 35/00; C07K 14/555; C07K 14/705; C12N 5/0786; C12N 9/12; C12N 11/08

(52) U.S. Cl. CPC A61K 35/15; A61K 39/4614; A61K 39/4631; A61K 39/464402; A61P 35/00; C07K 14/555; C07K 14/705; C12N 5/0645; C12N 9/1241; C12N 11/08; A61K 2239/15; A61K 2239/17; A61K 2239/21; A61K 2239/22

(57) ABSTRACT

A method for producing CAR-M1 macrophages expressing a chimeric antigen receptor in vitro and in vivo includes using a conjugate of a non-viral gene delivery system and a chimeric antigen receptor gene. The CAR-M1 macrophages are produced in vivo by delivering genes encoding a chimeric antigen receptor and IFN-γ, specifically to macrophages in the body, and thus does not require culturing and preparing an in-vitro cellular therapeutic agent, thus reducing the manufacturing costs of therapeutic agents. The CAR-M1 macrophages are a safer therapy since a non-viral vector is used, as compared to the production of CAR-M1 macrophages by gene delivery using a viral vector, and are a novel therapeutic candidate having the advantage of high anticancer efficiency for solid cancers, due to CAR-M1 macrophages in which intrinsic properties of macrophages infiltrating solid cancers and cancer cell phagocytosis are improved.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/003008, filed Mar. 3, 2022, which claims priority to the benefit of Korean Patent Application Nos. 10-2021-0028253 filed on Mar. 3, 2021 and 10-2022-0027426 filed on Mar. 3, 2022 in the Korean Intellectual Property Office, the entire contents of which are incorporated herein by reference.

### BACKGROUND

### 1. Technical Field

The present invention relates to a method for producing chimeric antigen receptor-macrophages and use of the same.

### 2. Background Art

Cancer immunotherapy, which has recently been emerging, is a treatment method that more specifically removes cancer cells by utilizing the body's own immune system.

Among them, immune cell therapy is a method of treating cancer by reinforcing the anticancer function of immune cells obtained from the patient's blood and injecting them back into the patient's body.

In particular, the recently approved chimeric antigen receptor (CAR) T cell (hereinafter referred to as “cell”) treatment by the U.S. Food and Drug Administration uses CAR-T cells that are designed to recognize and remove specific cancer cells through genetic engineering of T cells obtained from a patient.

CAR-T cell therapeutic agent shows currently high reactivity and cure rate in blood cancer. However, because of limited therapeutic effects in solid cancer and long time and high costs required for producing CA-T cells, clinical application of CAR-T cell therapeutics is inhibited.

The limited therapeutic effects in solid cancer are because it is difficult for CAR-T cells to enter the cancer tissue due to the tumor microenvironment in solid cancer, and the suppressed immune response interferes with the anticancer function of CAR-T cells.

Further, CAR-T cells use a viral gene carrier in the manufacturing process. Therefore, there are risks such as the possibility of developing immunogenicity after CAR-T cells are injected into the patient, and the possibility that the transferred gene may additionally activate cancer-related genes in the CAR-T cells.

### SUMMARY

The present invention solves the above problems and has been devised in response to the above necessity. An object of the present invention is to provide a method for reducing the risk of the existing therapeutic agents occurred due to use of a viral gene delivery system, by manufacturing a conjugate of a non-viral gene delivery system and a CAR gene.

In addition, another object of the present invention is to provide a method for reducing costs and time required to produce CAR-expressing macrophages in vitro by delivering CAR genes directly into the body thus to induce CAR-macrophages in situ.

Further, another object of the present invention is to provide a CAR macrophage-based therapeutic agent having high anticancer therapeutic effects in solid cancer.

The present invention provides a pharmaceutical composition for treating or preventing cancer, which includes a complex of a plasmid DNA including a gene which encodes a chimeric antigen receptor (CAR) and a non-viral carrier, or CAR macrophages transformed by the complex.

The plasmid DNA includes a chimeric antigen receptor (CAR) gene.

The chimeric antigen receptor (CAR) may include an antigen-binding domain to be bound to a disease antigen.

The disease antigen may be an antigen that is overexpressed in cancer cells. This may include, for example, anaplastic lymphoma kinase (ALK), CD19, HER2, CD22, CD30, CD73, CD123, FLT3, B-cell maturation antigen, PD-1 MUC16, MSLN, gp100, MART-1, Melna-A, MAGE-A3, MAGE-C2, Mammaglobin-A, proteinsase-3, mucin-1, HPV E6, LMP2, PSMA, GD2, hTERT, PAP, ERG, NA17, GM3, EPhA2, NA17-A, TRP-1, TRP-2, NY-ESO-1, CEA, CA 125, AFP, Survivin, AH1, ras, G17DT, MUC1, E75, p53, PSA, HCG, PRAME, WT1, URLC10, VEGFR1, VEGFR2, E7 or the like.

The antigen-binding domain is a single-chain variable fragment (scFv) of an antibody which binds to the antigen, and may be used without limitation as long as it is the scFv of an antibody against a target disease antigen.

A chimeric antigen receptor (CAR) may further include a hinge region, a transmembrane domain, a costimulatory domain or a signaling domain in addition to the antigen-binding domain.

A known configuration may be used for each region and domain of the CAR without limitation thereof.

As the hinge region, a hinge derived from CD8, CD28, etc. may be used, but it is not limited thereto.

As the transmembrane domain, a CD28-derived transmembrane domain may be used, but it is not limited thereto.

As the costimulatory domain, a costimulatory domain derived from CD28, CD27, CD134, CD137, etc. may be used, but it is not limited thereto. One or two or more thereof may be used.

As the signaling domain, a CD3-zeta-derived signaling domain may be used, but it is not limited thereto.

The CAR preferably further includes all or a part of the components described in FIG. 11, but it is not limited thereto.

The plasmid DNA may further include a gene encoding interferon-gamma (IFN-γ) to induce the M1 macrophage phenotype.

The origin of interferon gamma is not limited, and those derived from mice, humans, etc. may be used.

The complex may be formed by a combination of plasmid DNA and a non-viral carrier. For the combination, means, methods and combinations known in the art may be applied without limitation thereof.

The non-viral carrier refers to a non-viral gene carrier, not a viral carrier such as adenovirus. Any gene carrier that is not derived from a virus can be used without limitation thereof. In an aspect of easily forming the complex by electrostatic interaction with plasmid DNA, a cationic molecule is preferably used.

The cationic molecule refers to a molecule having a positive charge at neutral pH, and may have a nitrogen atom.

The cationic molecule may include, for example, polyethylene imine, mannosylated polyethyleneimine, mannosylated cholesterol-polyethyleneimine, PEG-polyethyleneimine-cholesterol, cationic lipids, methacrylate-based polymers including poly[(2-dimethylamino) ethyl methacrylate, polycations including chitosan and beta-cyclodextrin, polyamidoamines, dendrimers, degradable poly(β-ester), poly(lactic-co-glycolic acid), mannosylated liposomes, PEG-cholesterol-containing liposome and cationic ionizable lipid, PEG-cholesterol-containing liposomes and the like. In terms of targeting macrophages, preferably a mannosylated cationic molecule may be used, and more preferably, a mannosylated polyethyleneimine or a mannosylated liposome may be used.

The mannosylated polyethyleneimine is a polymer in which mannose is bound to the amino group of PEI. For example, α-d-Mannopyranosylphenyl isothiocyanate (MPITC) may be bound. PEI may be branched PEI, and a molecular weight thereof may be, for example, 1000 to 100000, 10000 to 50000, etc., but it is not limited thereto.

When the plasmid DNA and the non-viral carrier are combined through electrostatic attraction to form a complex, these may be transferred into macrophages and controlled to have an appropriate level of attraction so that the carrier can be released later. This may be controlled, for example, by adjusting a ratio of nitrogen (N) in the cationic molecule to phosphorus (P) in the plasmid DNA. For example, in the complex, a ratio (N/P, element ratio) of nitrogen (N) of the non-viral carrier cationic molecule to phosphorus (P) of the plasmid DNA may be 4 to 30. Within the above range, the ratio may be 4 to 30, 4 to 25, 4 to 20, 4 to 15, 4 to 10, 4 to 8 and the like.

The complex may further include a transposase plasmid. This may be, for example, a piggybac transposase plasmid.

The transposase plasmid may be included by mixing with plasmid DNA and a non-viral carrier during the preparation of the complex.

CAR macrophages are macrophages transformed with the complex to express CAR.

CAR macrophages express the CAR, and since the CAR may include an antigen-binding domain for a disease antigen, CAR macrophages may exhibit drug efficacy against the disease.

The macrophage may be, for example, bone marrow derived macrophage (BMDM) or a macrophage cell line (J774A.1, RAW 264.7, etc.), but it is not limited thereto.

CAR macrophages may be prepared by treating macrophages with the complex and transforming macrophages. The transformation may be performed in vivo or in vitro.

The cancer to be prevented or treated by the pharmaceutical composition of the present invention may be determined according to the type of the antigen-binding domain included in the CAR, and the type of the antigen-binding domain that can be introduced into the CAR is not limited, thus the type of the cancer is not limited. For example, the cancer may be any one of brain cancer, head and neck cancer, bladder cancer, breast cancer, cervical cancer, colon cancer, colorectal cancer, endometrial cancer, esophageal cancer, leukemia, lung cancer, liver cancer, ovarian cancer, pancreatic cancer, prostate cancer, rectal cancer, kidney cancer, stomach cancer, testicular cancer, uterine cancer, vascular tumor, squamous cell carcinoma, adenocarcinoma, small cell carcinoma, melanoma, glioma, neuroblastoma, sarcoma, laryngeal cancer, parotid cancer, biliary tract cancer, thyroid cancer, actinic keratosis, acute lymphocytic leukemia, acute myeloid leukemia, adenocystic carcinoma, adenoma, adenomatous squamous carcinoma, anal duct cancer, anal cancer, anorectal cancer, astrocytoma, greater vestibular gland cancer, basal cell carcinoma, cholangiocarcinoma, bone cancer, bone marrow cancer, bronchial cancer, bronchial adenocarcinoma, carcinoid, cholangiocarcinoma, chronic lymphocytic leukemia, chronic myelogenous leukemia, clear cell carcinoma, connective tissue cancer, cystic adenoma, digestive system cancer, duodenal cancer, endocrine system cancer, endoderm sinus tumor, endometrial hyperplasia, endometrioid adenocarcinoma, endothelial cell carcinoma, ependymal cell carcinoma, epithelial cell carcinoma, orbital cancer, focal nodular hyperplasia, gallbladder cancer, pyelonephroma, gastric basal cancer, gastrinoma, glioblastoma, neuroblastoma, glucagonoma, heart cancer, hemangioblastoma, hemangioendothelioma, hemangioma, hepato adenoma, liver cell adenoma, hepatobiliary tract cancer, hepatocellular carcinoma, Hodgkin's disease, ileal cancer, insulinoma, intraepithelial neoplasia, intraepithelial squamous cell neoplasia, intrahepatic biliary tract cancer, invasive squamous cell carcinoma, jejunum cancer, joint cancer, pelvic cancer, giant cell carcinoma, colorectal cancer, lymphoma, malignant mesothelial cell tumor, medulloblastoma, medullary epithelioma, meningeal cancer, mesothelial cancer, metastatic carcinoma, oral cancer, mucoepidermoid carcinoma, multiple myeloma, muscle cancer, nasal duct cancer, nervous system cancer, non-epithelial skin cancer, Non-Hodgkin's lymphoma, soft cell carcinoma, oligodendroglioma cancer, mouth cancer, osteosarcoma, papillary serous adenocarcinoma, penile cancer, pharyngeal cancer, pituitary tumor, plasmacytoma, pseudosarcoma, pulmonary blastoma, rectal cancer, renal cell carcinoma, respiratory system cancer, Retinoblastoma, serous carcinoma, sinus cancer, skin cancer, small cell carcinoma, small intestine cancer, smooth muscle cancer, soft tissue cancer, somatostatin-secreting tumor, spinal cancer, epithelial squamous cell cancer, striatal muscle cancer, submesothelial carcinoma, T cell leukemia, tongue cancer, ureter cancer, urethral cancer, cervical cancer, uterine trunk cancer, vaginal cancer, VIPoma, vulvar cancer, highly differentiated carcinoma and Wilm's tumor.

The pharmaceutical composition of the present invention may include pharmaceutically acceptable carriers, that is, saline, sterile water, Ringer's solution, buffered saline, cyclodextrin, dextrose solution, maltodextrin solution, glycerol, ethanol, liposome, and a mixture of one or more of these components, and may further include other conventional additives, such as antioxidants and buffers, if necessary. Further, diluents, dispersants, surfactants, binders and/or lubricants may be additionally added to form an injectable formulation such as an aqueous solution, suspension, emulsion, etc., pills, capsules, granules or tablets.

Furthermore, the pharmaceutical composition may be formulated according to individual components by an appropriate method known in the art or according to a method disclosed in Remington's Pharmaceutical Science, Mack Publishing Company, Easton PA. The pharmaceutical composition of the present invention is not particularly limited in formulation, but is preferably formulated as an injection or inhalant.

The administration method of the pharmaceutical composition according to the present invention is not particularly limited, but may include non-parenteral administration such as intravenously, subcutaneously, intraperitoneally, inhalation or topical application, or oral administration, depending on the desired method. The dosage of the pharmaceutical composition of the present invention may vary widely depending on the weight, age, sex, health conditions or diet of the patient, administration time, administration method, excretion rate and severity of the disease.

The daily dose refers to an amount of a therapeutic substance according to one aspect sufficient to treat a disease state alleviated by being administered to a subject in need of treatment. The effective amount of a therapeutic agent will depend on the particular compound, the disease state and its severity, and the individual in need of treatment, which can be routinely determined by those skilled in the art. As a non-limiting example, the dosage for the human body of the composition according to one aspect may vary depending on the patient's age, weight, sex, dosage form, health status, and disease degree. Based on an adult patient weighing 70 kg, for example, about 1,000-10,000 cells/time, 1,000-100,000 cells/time, 1,000-1,000,000 cells/time, 1,000-10,000,000, 1,000-100,000,000 cells/time, 1,000 to 1,000,000,000 cells/time, 1,000 to 10,000,000,000 cells/time, may be administered in divided doses once or several times a day at a regular time interval, or may be administered several times at a regular time interval.

The present invention also relates to a method for production of CAR macrophages, which includes transforming the macrophages with a complex of a plasmid DNA including a gene which encodes a chimeric antigen receptor and a non-viral carrier.

The plasmid DNA including a gene encoding a chimeric antigen receptor, the non-viral carrier, the complex thereof, and the macrophage may be within the scope of the above-described examples.

The transformation may be performed by treating the macrophages with the complex, and the transformation method, means, conditions, etc. may be as known in the art.

The transformation may be performed in vivo or in vitro. When performed in vivo, the complex may be administered to an individual, and the complex may be in contact with macrophages in the body. When performed in vitro, it may be carried out by treating macrophages with the complex.

The present invention may further include preparing the complex by mixing the plasmid DNA and the non-viral carrier in a ratio of Equation 1 below.

4≤N/P≤30 [Equation 1]

(Wherein, N is nitrogen of the non-viral carrier cationic molecule, P is phosphorus of the plasmid DNA, and the ratio is the number of elements).

When Equation 1 is satisfied, the plasmid DNA and the non-viral carrier are combined through an appropriate attractive force, so that it is easy to deliver the combination to macrophages and then eliminate the viral carrier. Within the above range, it may be 4 to 30, 4 to 25, 4 to 20, 4 to 15, 4 to 10, 4 to 8 and the like.

The present invention also provides a method for providing information for diagnosis of cancer, which includes contacting a CAR macrophage with a sample isolated from a subject.

CAR macrophages are macrophages transformed with the above-described complexes.

CAR macrophages bind to the cancer cells having an antigen which binds to the antigen-binding domain, and according to the binding, it is possible to know whether the cancer cells are present or not, and thereby providing information on whether the subject gets cancer or not.

The present invention provides an effective immunocancer treatment means that produces a CAR gene conjugate using a non-viral gene delivery system and delivers it specifically to macrophages in vivo.

The present invention will also be a technique for ensuring stability from immune response using a non-viral gene delivery system. Further, it is advantageous in terms of commercialization in that it reduces the complicated in vitro therapeutic manufacturing process by directly transferring the gene in vivo, while reducing the economic burden on the patient by decreasing the production costs, and may be administered directly to the patient when the therapeutic agent is needed.

The present invention is also effective to contribute to extend the range of cancer types to which anticancer effects of CAR-based therapeutics as a treatment agent that shows anticancer effects in solid cancer can be applied, unlike CAR-T cells as existing anticancer drugs, which have little therapeutic effects in solid cancer due to cancer-friendly effect in tumor microenvironment.

### BRIEF DESCRIPTION OF THE DRAWINGS

FIGS. 1A and 1B show confirmation of the insertion of pCAR-IFN-g genes on the non-viral piggybac plasmid vector and the viral pCDH plasmid vector by double digestion through treatment with two types of restriction enzymes, wherein, in the case of the piggybac plasmid vector, it was delivered to BMDM by mixing with M PEI, while, in the case of pCDH plasmid vector, a lentivirus was made and treated in BMDM at each concentration, followed by transduction confirmed 72 hours later.

FIG. 2 shows results of confirming the expression of anapiastic lymphoma kinase (ALK) gene by a reverse transcription polymerase chain reaction using intracellular mRNA, wherein two types of ALK gene cDNA-specific primers were prepared through organic synthesis.

FIG. 3 shows results of confirming that ALK overexpression occurs in most neuroblastomas based on previous studies. Therefore, in order to confirm ALK overexpression in neuroblastoma, the expression of the target antigen ALK gene in B16F10 (ATCC), NIH/3T3 (ATCC) and Neuro2A (ATCC) cell lines were investigated for qualification and quantification of ALK gene expression level on Neuro2A as one of the target carcinomas, so as to confirm overexpression of neuroblastoma-specific ALK on Neuro2A through the reverse transcription polymerase chain reaction.

FIG. 4 shows an experimental procedure to determine ALK-specific cell apoptosis ability and phagocytosis of macrophages expressing CAR gene through co-culture with the B16F10 cell line (ATCC) not expressing ALK gene, the NIH/3T3 cell line (ATCC), or the Neuro2a cell line (ATCC) expressing the ALK gene.

FIG. 5 shows results of confirming the apoptosis-inducing response over time by live cell imaging using PAULA (Leica microsystems) at a 10-minute interval after co-culture with the Neuro2a cell line (ATCC) expressing the ALK gene for 16 hours.

FIG. 6 shows analysis of a ratio of living cancer cells through flow cytometry analysis after co-culture was performed for 24 hours by varying a ratio of BMM to the cancer cells, wherein it was confirmed that the number of living cancer cells was decreased in the situation where the CAR gene was expressed.

FIG. 7 shows results of confirming that phagocytosis is increased in co-culture of CAR and GFP co-expressing cells by gene transfer with Neuro2a cells expressing ALK after co-culture of BMM and cancer cells with E:T ratio of 1:3 for 24 hours.

FIG. 8 is a diagram confirming that the number of cancer cells is reduced in the situation where the CAR gene is expressed after co-culture was performed for 48 hours by varying a ratio of RAW264.7 cell line to the cancer cells,

wherein it was confirmed that the ratio of living Neuro2 cells expressing ALK to CAR expressing cells is reduced.

FIG. 9 shows a picture confirming that phagocytosis is increased in the situation where the CAR gene is expressed after co-culture was performed by varying the ratio of RAW264.7 cell line to the cancer cells for 48 hours,

wherein it was confirmed that phagocytosis is increased in the co-culture of CAR expressing cells and ALK expressing Neuro2a cells.

FIG. 10 shows confirmation of the change in phagocytosis by gene transfer after co-culture of the RAW 264.7 cell line and cancer cells was performed at E:T ratio 1:3 for 24 hours.

FIG. 11 is a schematic diagram of the vector plasmid construct of the present invention.

FIG. 12 shows results of confirming that CAR expression was highest in cancer tissues relative to other organs in mice injected with MPEI/pCAR-IFN-γ, as compared to the group injected with only the solution containing nanoparticles (Vehicle), wherein CAR was most expressed in macrophages compared to other immune cells (dendritic cells, B cells and T cells) in cancer tissues,

FIG. 13 shows results of observing that CAR expressing macrophages in cancer tissues (green in FIG. 13) overlap with or close to the cancer cells (red in FIG. 13) in terms of fluorescence through immunohistochemical analysis, thereby confirming that CAR expressing macrophages prey on the cancer cells.

FIG. 14 shows confirmation of the changes in liver and kidney-related enzymes [aspartate aminotransferase (AST) and alanine aminotransferase (ALT), creatinine and blood urea nitrogen (BUN)] in plasma by extracting blood for each time period from mice treated with nanoparticles, therefore, confirming that the difference between the corresponding enzymes in each plasma of mice injected with vehicle and mice injected with MPEI/pCAR-IFN-γ was not statistically significant.

FIGS. 15 and 16 show results of observing that: when comparing the control group with a group injected with only a solution containing nanoparticles (Vehicle), a nanoparticle group containing a gene which is not a CAR-M1 inducer (MPEI/Mock), and a nanoparticle group containing only the CAR gene (MPEI/pCAR), and a nanoparticle group containing the M1-inducer alone (MPEI/pIFN-γ), the cancer growth was statistically and significantly inhibited in the group treated with MPEI/pCAR-IFN-γ; and a survival rate of mice was increased.

FIGS. 17 and 18 show results of confirming that: a ratio of cell nucleus to cytoplasm was decreased in the MPEI/pCAR-IFN-γ-treated group in which the growth of cancer cells was most inhibited; and the most apoptotic cells were observed through hematoxylin and eosin (H&E) staining and TUNEL assay.

FIGS. 19, 20 and 21 show results of confirming that the number or function of immune cells related to anticancer function in mice after MPEI/pCAR-IFN-γ treatment was significantly increased through qRT-PCR, flow cytometry and immunohistochemical analysis.

FIG. 22 shows results of confirming that: the number and function of activated cytotoxic T cells attacking cancer were increased in the group injected with MPEI/pCAR-IFN-γ compared to other groups through flow cytometry; an amount of regulatory T cells (Treg) interfering with anticancer immunity was most decreased; and activation of anticancer immunity was demonstrated through the largest ratio of CD8+ T cells/Treg, which is an indicator of immune activation.

FIG. 23 shows results of confirming that a TGF-β⁺ area involved in the differentiation of Tregs, which contributes to cancer growth in cancer tissues, was reduced when treated with the groups including pIFN-γ (MPEI/pIFN-γ and MPEI/pCAR-IFN-γ).

FIG. 24 shows results of confirming that an amount of inflammatory factors (TNF-α and IFN-γ) involved in the anticancer function in the cancer tissue was increased, while the amount of factors (IL-4 and IL-10) involved in inhibiting the anticancer function was decreased.

FIG. 25 shows results of confirming that, even when MPEI/pCAR-IFN-γ was intraperitoneally injected, a significant number of nanoparticles were accumulated in cancer, and CAR was expressed in macrophages in cancer tissues.

FIG. 26 shows results of observing that macrophages expressing CAR in cancer tissues [green in FIG. 26] prey on phagocytic cancer cells [red in FIG. 26] through immunohistochemical analysis.

FIG. 27 shows results of confirming that MPEI/pCAR-IFN-γ injected intraperitoneally had cancer growth inhibitory effects similar to that directly injected into the cancer.

FIG. 28 shows results of confirming that MPEI/pCAR-IFN-γ does not have significant toxicity in vivo compared to the control group even after intraperitoneal injection into cancer-causing mice.

FIG. 29 shows efficiencies of in vitro transformation (CAR gene expression) of macrophages into CAR macrophages by adding a complex of CAR/IFN-γ/GFP plasmid DNA and cationic liposomes to in vitro macrophage culture.

FIG. 30 shows efficiencies of in vivo transformation (CAR gene expression) of macrophages present in the abdominal cavity into CAR macrophages by injecting a complex of CAR/IFN-γ/GFP plasmid DNA and cationic liposome into the abdominal cavity of a mouse.

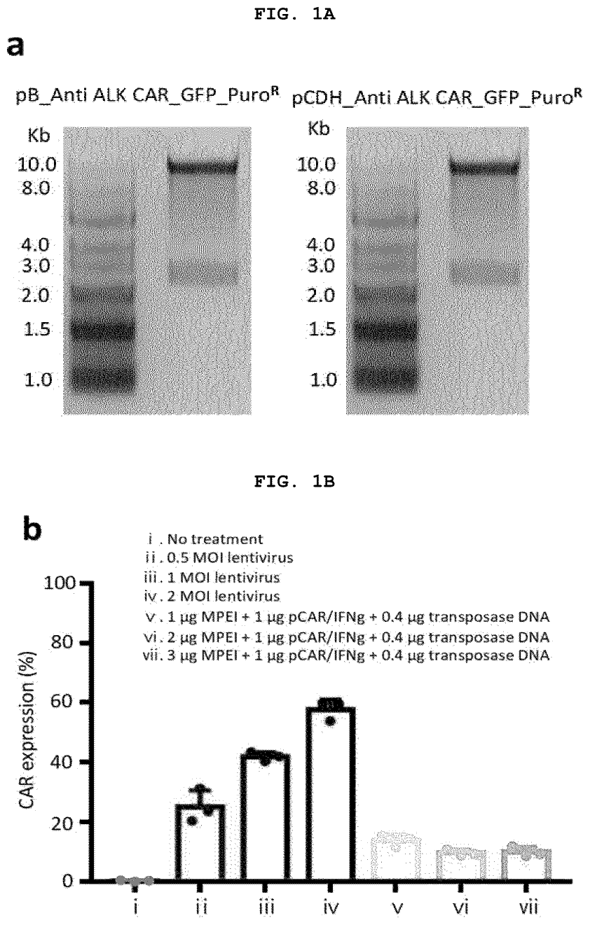

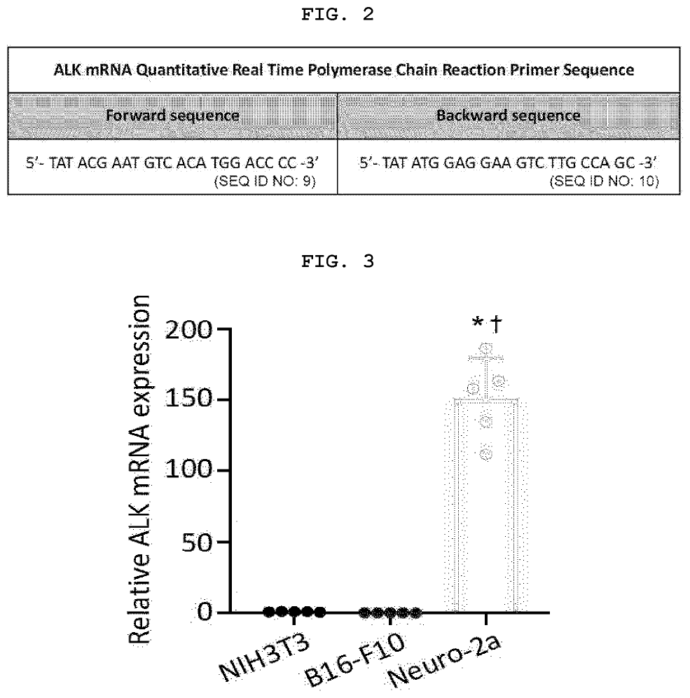

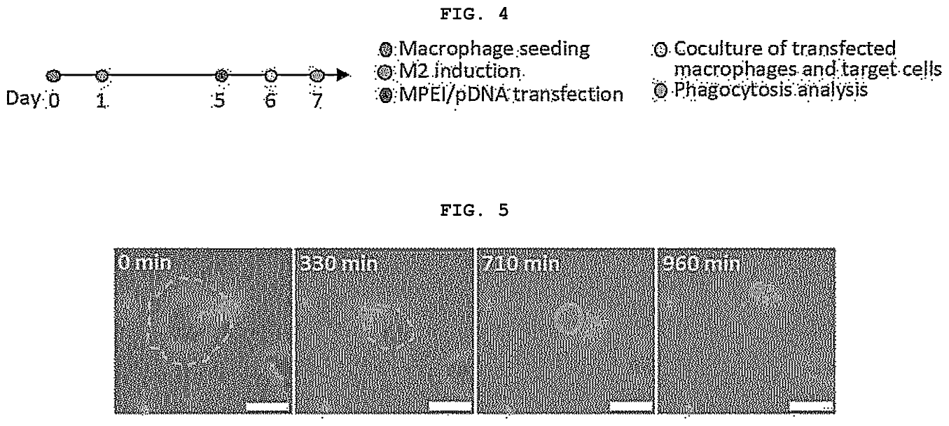

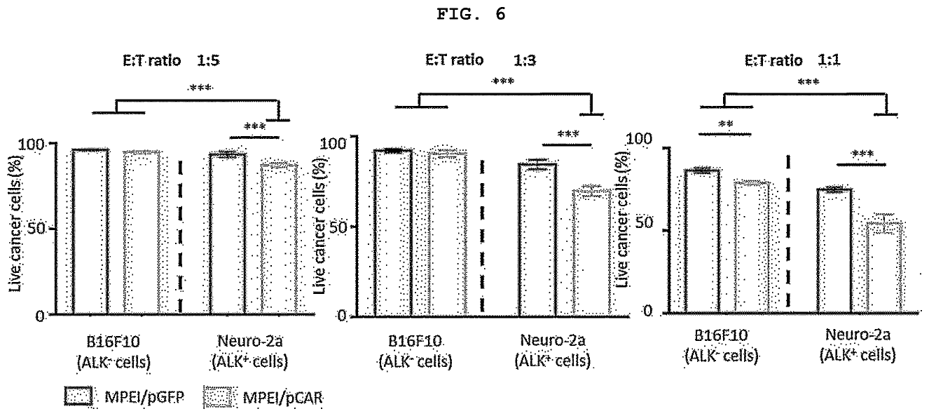

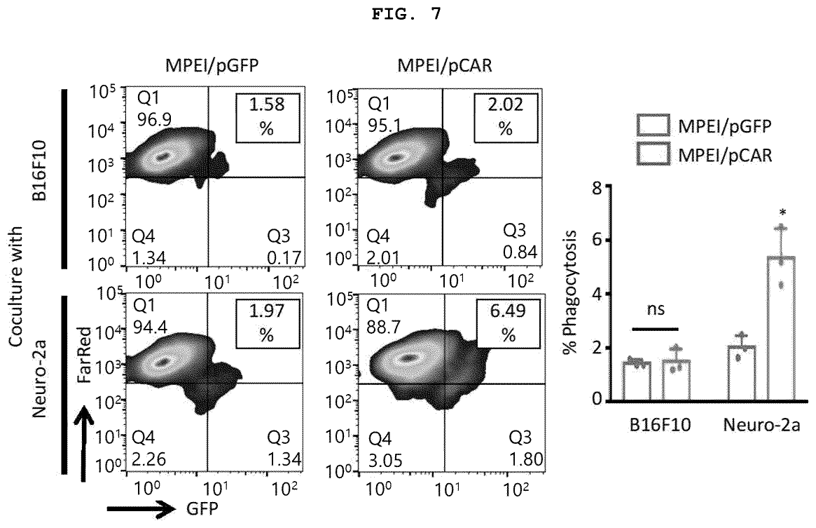

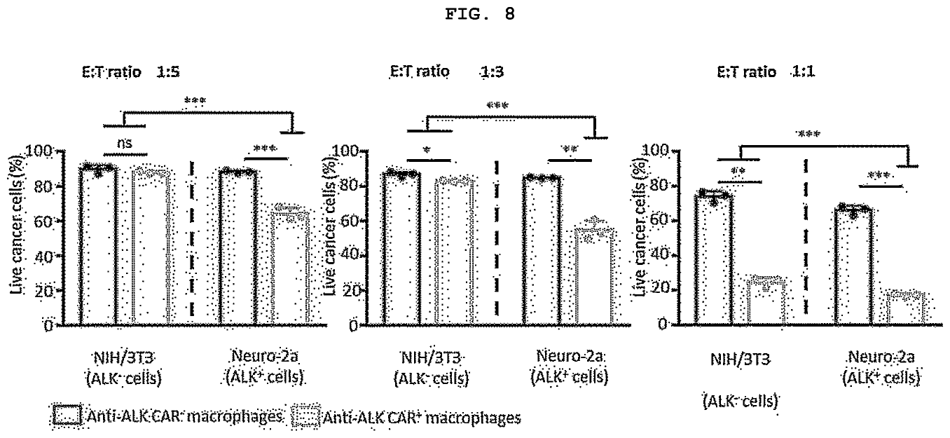

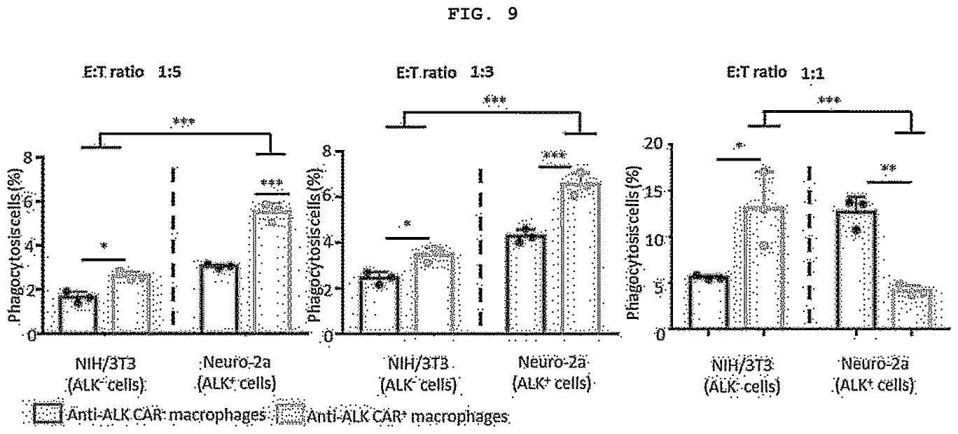

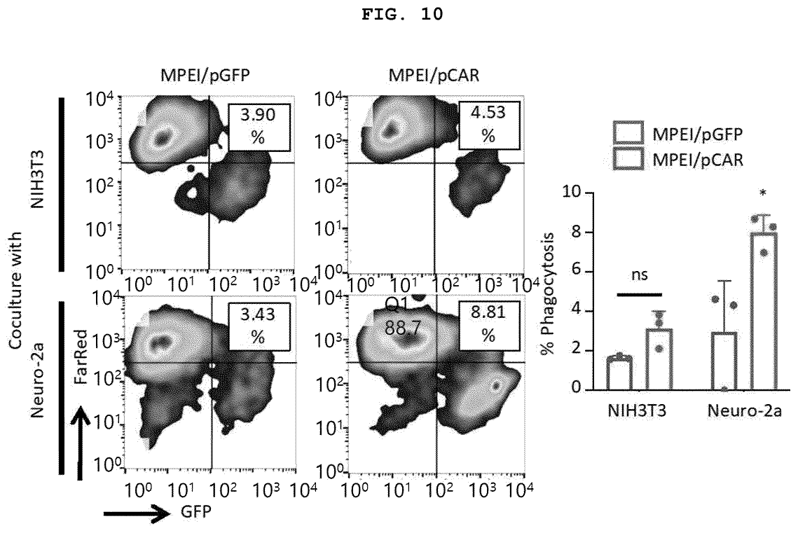

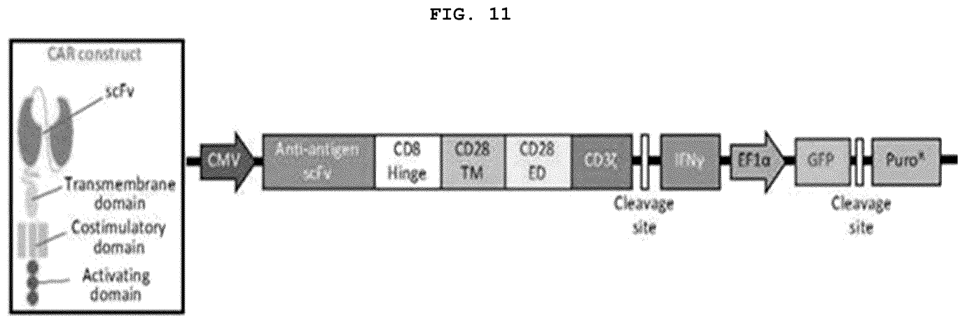

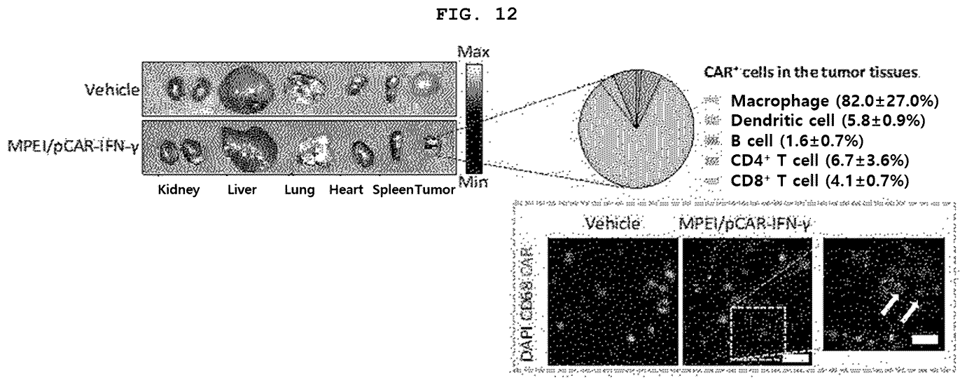

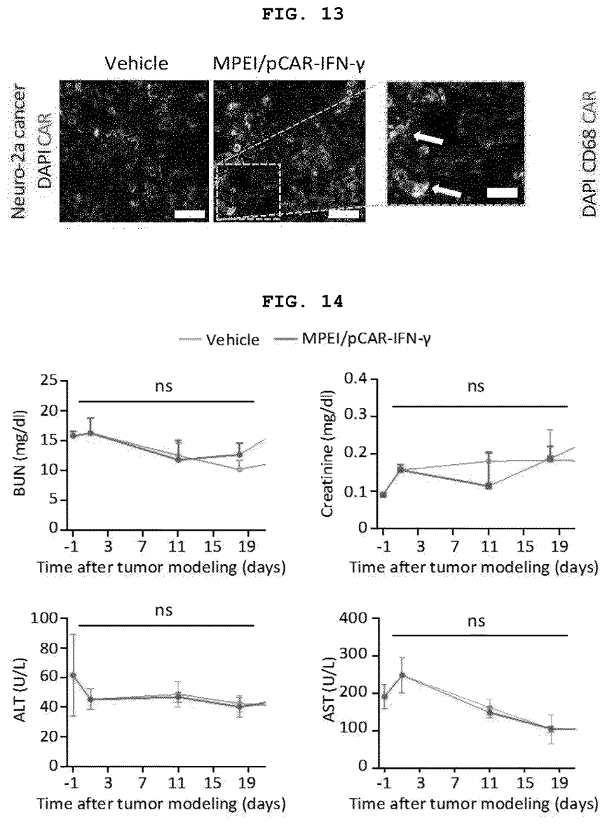

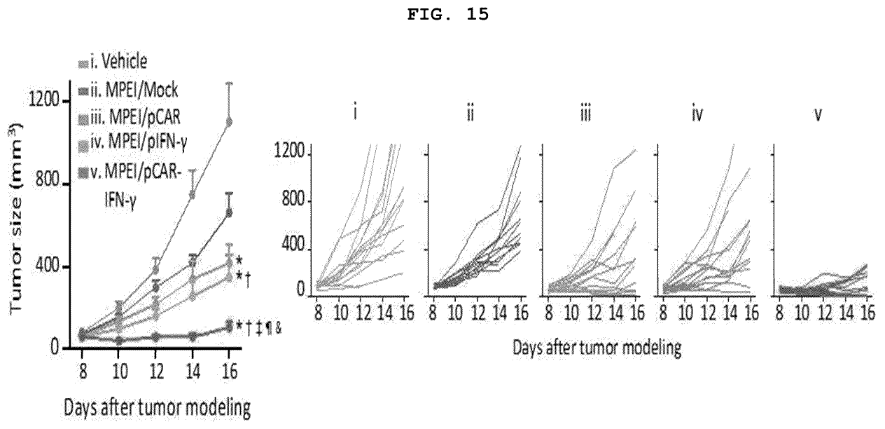

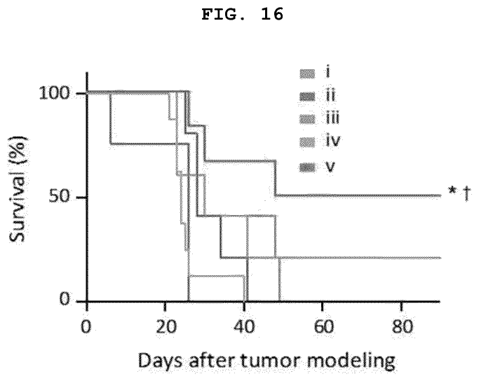

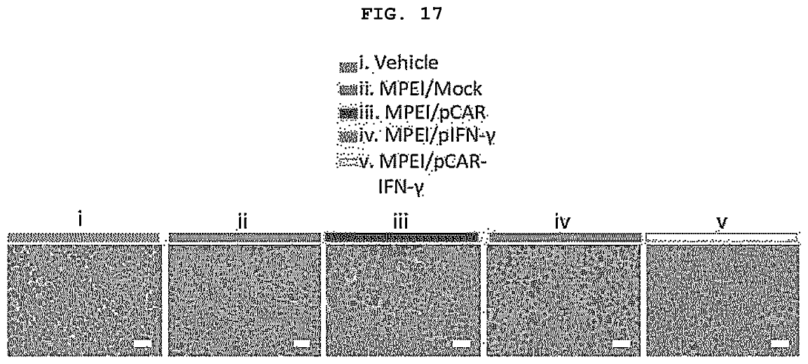

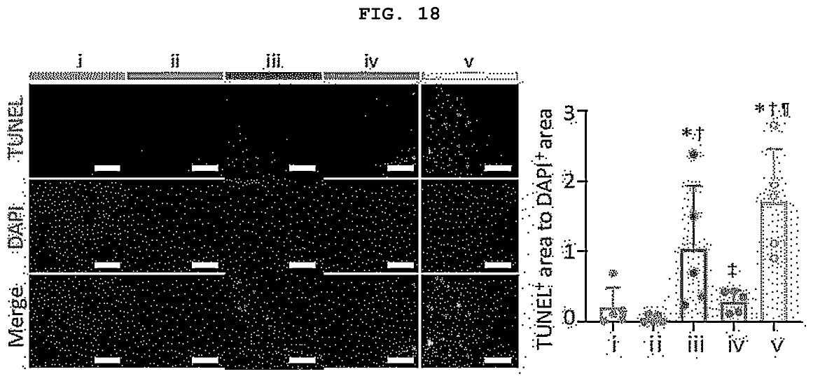

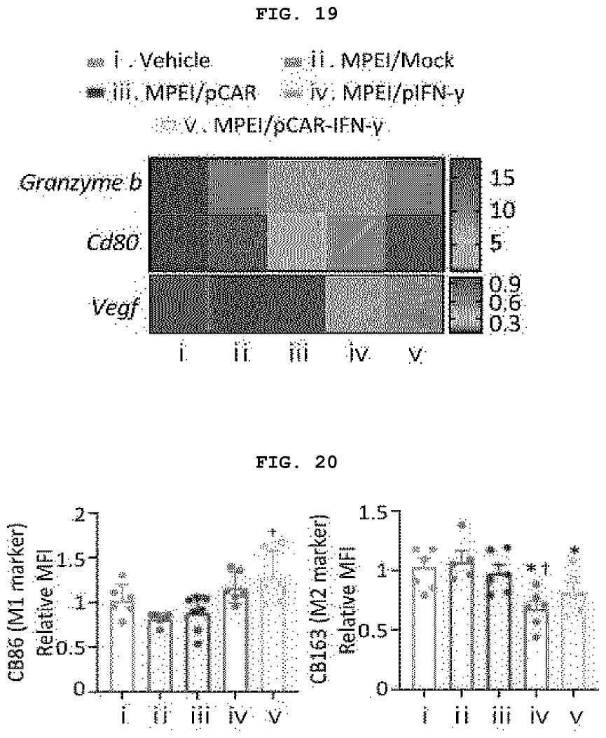

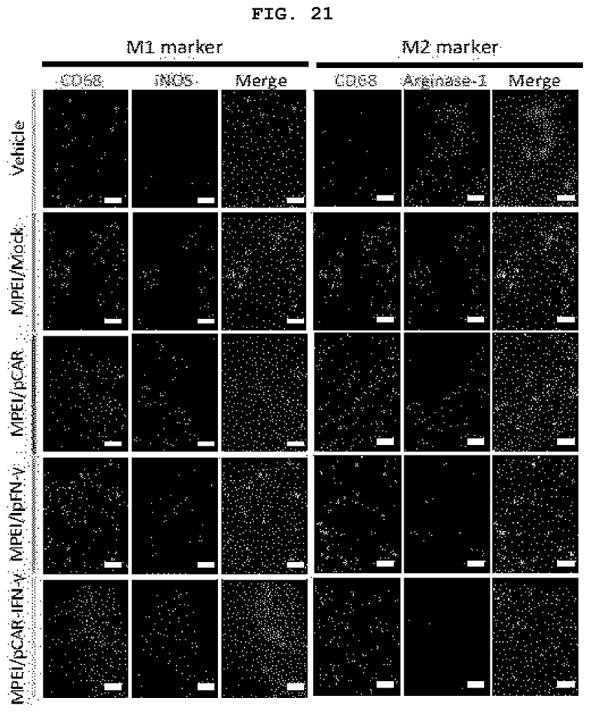

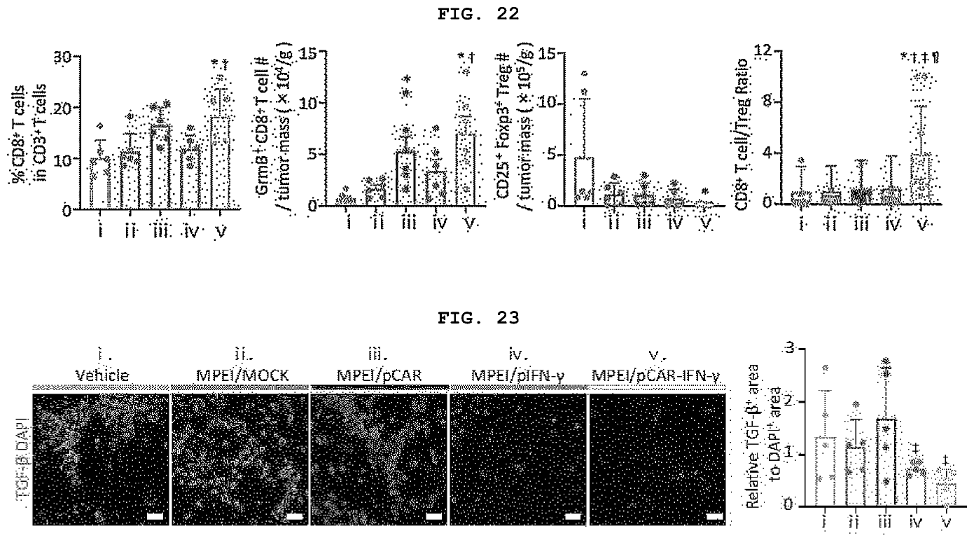

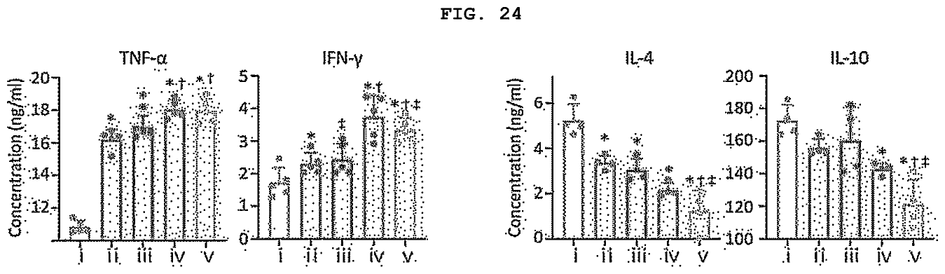

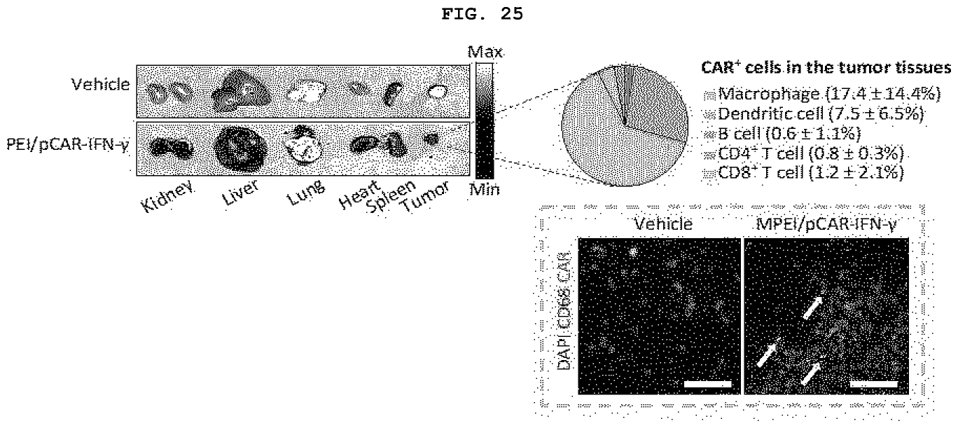

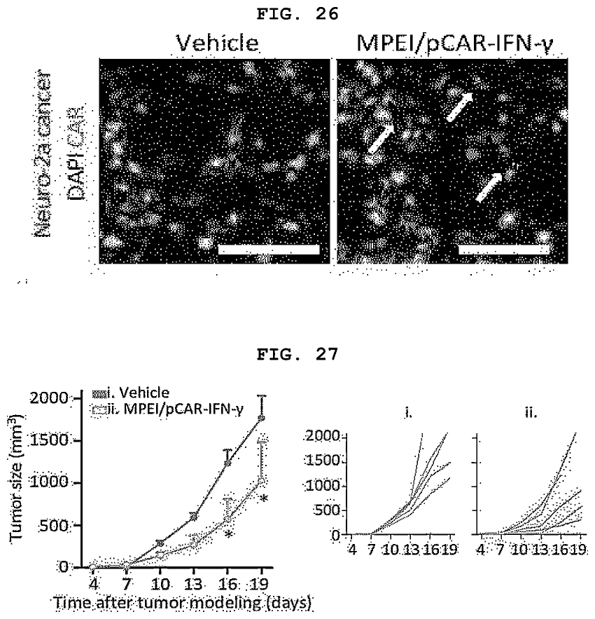

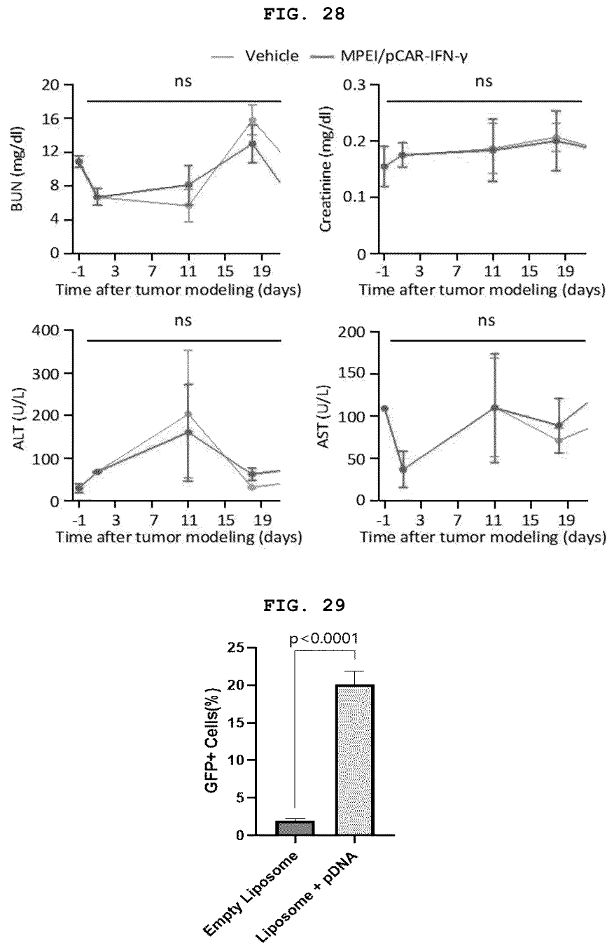

### DETAILED DESCRIPTION

Hereinafter, the present invention will be described in more detail through non-limiting examples. However, the following examples are intended to illustrate the present invention, and the scope of the present invention is not to be construed as being limited by the following examples.

### Example 1

Anti-anaplastic lymphoma kinase (ALK) chimeric antigen receptor (hereinafter, CAR) genes (CD8a SP of SEQ ID NO: 1, ALK scFv of SEQ ID NO: 2, CD8a hinge of SEQ ID NO: 3, CD28 transmembrane domain of SEQ ID NO: 4, CD28 cytoplasmic domain of SEQ ID NO: 5, CD3z (CD247) cytoplasmic domain of SEQ ID NO: 6, linker of SEQ ID NO: 7, using cleavage sequence) were synthesized by using a gene organic synthesis method, inserted into pUC-AMP vector, and amplified, followed by amplifying CAR gene inserted in the pUC-AMP vector through PCR and then isolating the same. Thereafter, the isolated CAR gene was inserted into the MCS sequence of lentiviral pCDH-CMV-MCS-EF1α-copGFP Cloning and Expression Lentivector purchased from SBI (system biosciences) through an infusion method, followed by cloning the same thus to complete the pCDH_CAR plasmid.

A mouse IFNg sequence (SEQ ID NO: 8) present in Interferon Gamma cDNA ORF Clone, Mouse, untagged plasmid purchased from Sino biological was extracted by double digestion using two restriction enzymes KpnI and XbaI, then the product was inserted and cloned on pCDH_CAR plasmid at 3′ end of CAR gene through PCR cloning, thus to complete pCDH_CAR_IFN-γ.

In order to produce a lentivirus containing pCDH_CAR plasmid (or pCDH_CAR_IFN-γ), pCDH_CAR plasmid (or pCDH_CAR_IFN-γREV), lenti G/P, and VSV-G were mixed with PEI (25 k linear, poly ethylene imine), and stored at room temperature for 30 minutes, followed by treating the product to a lenti-X 293T cell line (TaKaRa bio) for 8 hours.

After 72 hours, the culture medium was filtered with a 0.45 um filter, and centrifuged at 4° C. for 2 hours at a speed of 20000 RPM. The resulting lentivirus pallet was concentrated and diluted to a volume of 1/100.

After adding the concentrated pCDH_CAR plasmid (or pCDH_CAR_IFN-γ)-containing lentivirus to a complete DMEM, high glucose (supplemented with 10% FBS, 1% pen-strep and 4 ug/ml polybrene) at a concentration of 3 MOI, and then added to RAW 264.7 cell line for 24 hours.

After 72 hours of lentivirus treatment, pCDH_CAR plasmid (or pCAR RAW 264.7 cell line transduced with pCDH_CAR_IFN-γ (or pCAR IFN-γ RAW 264.7 cell line)) was isolated and investigated through cell line selection.

### Example 2

Anti-anaplastic lymphoma kinase (ALK) chimeric antigen receptor (hereinafter, CAR) genes were synthesized by using a gene organic synthesis method, inserted into pUC-AMP vector, and amplified, followed by amplifying CAR gene inserted in the pUC-AMP vector through PCR and then isolating the same. Thereafter, the isolated CAR gene was inserted into MCS sequence of a non-viral PB-CMV-MCS-EF1α-GreenPuro PiggyBac cDNA purchased from SBI (system biosciences) through an infusion method, followed by cloning the same thus to complete the pB_CAR plasmid.

A mouse IFNg sequence present in Interferon Gamma cDNA ORF Clone, Mouse, untagged plasmid purchased from Sino biological company was extracted by double digestion using two restriction enzymes KpnI and XbaI, then the product was inserted and cloned on pB_CAR plasmid at 3′ end of CAR gene through PCR cloning, thus to complete PB_CAR_IFN-γ (hereinafter, pCAR-IFN-γ).

Mannosylated polyethyleneimine (PEI) (Jet PEI-Mac, polyplus-transfection, hereinafter MPEI), pCAR (or pCAR-IFN-γ) and piggybac transposase plasmid were mixed and stored at room temperature for 20 minutes, followed by treating the product to M2 BMDM (bone marrow derived macrophage) for 2 hours.

After 72 hours, expression of pCAR (or pCAR-IFN-γ gene) was confirmed.

An increase in antigen-specific apoptosis capacity and phagocytosis of BMDM and RAW 264.7 cell lines expressing the pCAR (or pCAR-IFN-γ) gene was observed.

### Example 3

Pure mRNA was isolated from B16F10 (ATCC), NIH/3T3 (ATCC) and Neuro2A (ATCC) using Trizol (Qiagen, Valencia, CA)

The mRNA amount isolated from B16F10 (ATCC), NIH/3T3 (ATCC) and Neuro2A (ATCC) was quantified using a NanoDrop spectrometer (ND-2000, NanoDrop Technologies). Then, a certain amount of mRNA was diluted in ACCUPOWER®MASTER Mix (Bioneer) by 1:1, incubated at 70° C. for 5 minutes, and stored on ice, followed by treating the same at 42° C. for 60 minutes to synthesize cDNA. Thereafter, the product was treated at 94° C. for 5 minutes to inactivate RTase.

cDNA derived from B16F10 (ATCC), NIH/3T3 (ATCC), Neuro2A (ATCC) were mixed with SYBR green-based TOPreal™ qPCR 2X PreMIX (Enzynomics), 10 pmol/μl Forward primer, and 10 pmol/μl Reverse primer. After treatment at 95° C. for 15 minutes, the three-stage treatment process consisting of 10 seconds at 95° C., 15 seconds at 62° C., and 20 seconds at 72° C. was repeated 55 times to confirm the expression of ALK in real time.

### Example 4

After mixing MPEI, pCAR-IFN-γ and piggybac transposase plasmid, the mixture was stored at room temperature for 20 minutes, and then treated to M2 BMDM (bone marrow derived macrophage) for 2 hours.

Then, after 24 hours, StemPro Accutase Cell Dissociation Reagent (Thermo-Fisher Scientific) was treated at room temperature for 10 to 15 minutes to float MPEI/pCAR-IFN-γ-treated M2 BMDM, followed by centrifugation at 1500 RPM for 5 minutes to obtain cell pellets. Then, the cell pellets were resuspended in RPMI 1640 (Gibco, supplemented 10% FBS, 1% pen-strep, 20 ng/ml M-CSF).

Thereafter, M2 BMDM treated with MPEI/pCAR-IFN-γ was stained using CELLTRACE′M Cell Proliferation Kit (thermofisher scientific).

BMDM stained with CELLTRACE′M Cell Proliferation Kit (Thermo-Fisher Scientific) and Neuro2A (ATCC) expressing ALK gene were treated at a concentration of 1:3 in a 24-well plate (SPL), and co-culture was performed for 16 hours.

At this time, the co-cultured cells were observed up to 16 hours by time-lapse at a 10-minute interval using PAULA (Leica Microsystems).

As a result of observation under co-culture conditions, BMDM (green) treated with MPEI/pCAR-IFN-γ completely removed Neuro2A (ATCC, white) from the bottom surface after 710 minutes, and at 960 minutes, it was confirmed that apoptosis was induced as Neuro2A (ATCC) cells removed from the bottom surface were reduced.

### Example 5

After mixing MPEI, pCAR, and piggybac transposase plasmid, the mixture was stored at room temperature for 20 minutes, and then treated in M2 BMDM (bone marrow derived macrophage) for 2 hours.

Then, after 24 hours, StemPro Accutase Cell Dissociation Reagent (Thermo-Fisher Scientific) was treated at room temperature for 10 to 15 minutes to float MPEI/pCAR-treated M2 BMDM, followed by centrifugation at 1500 RPM for 5 minutes thus to obtain cell pellets. Then, the cell pellets were resuspended in RPMI 1640 (Gibco, supplemented 10% FBS, 1% pen-strep, 20 ng/ml M-CSF).

Thereafter, MPEI/pCAR-treated M2 BMDMs were stained using CELLTRACE™ Cell Proliferation Kit (Thermo-Fisher Scientific).

BMDM stained with CELLTRACE™ Cell Proliferation Kit (Thermo-Fisher Scientific) and Neuro2A (ATCC) expressing ALK gene were treated at a concentration of 1:3 in a 24-well plate (SPL), and co-culture was performed for 24 hours.

As a result of the analysis, it was observed that a cell ratio of Neuro2A (ATCC) was decreased in the co-culture condition with MPEI/pCAR-treated M2 BMDM and ALK-expressing Neuro2a. Further, although the number of Neuro2A(ATCC) was most reduced at E:T ratio of 1:1 in the co-culture condition with Neuro2A (ATCC), effective results were demonstrated in terms of a decrease range in cell ratio of NIH/3T3 (ATCC) by M2 BMDM treated with MPEI/pCAR in the co-culture condition with NIH/3T3 (ATCC), as compared to the cell ratio of NIH/3T3 (ATCC) by M2 BMDM treated with MPEI/pGFP in the co-culture condition with NIH/3T3 (ATCC).

It was observed that the cell ratio of Neuro2A (ATCC) was decreased in the co-culture condition with MPEI/pCAR-treated M2 BMDM and ALK-expressing Neuro2a, the number of Neuro2A (ATCC) at E:T ratio of 1:3 in the co-culture condition with Neuro2A (ATCC), was reduced by about 25%, and ineffective results were demonstrated in terms of a decrease range in cell ratio of NIH/3T3 (ATCC) by M2 BMDM treated with MPEI/pCAR in the co-culture condition with NIH/3T3 (ATCC), as compared to the cell ratio of NIH/3T3 (ATCC) by M2 BMDM treated with MPEI/pGFP in the co-culture condition with NIH/3T3 (ATCC).

Therefore, it can be seen that the ratio of Neuro2A (ATCC) cells is decreased as the ratio of BMDM as an effector cell is increased at E:T ratio. Accordingly, it was confirmed that the decrease range in the ratio of Neuro2a (ATCC) cells was specifically shown at E:T ratio of 1:3.

It was observed that the cell ratio of Neuro2A (ATCC) was decreased in the co-culture condition with the MPEI/pCAR-treated M2 BMDM and the ALK-expressing Neuro2a. Further, it was confirmed that the number of cells with phagocytosis activity at E:T ratio of 1:3 in the co-culture condition with Neuro2A (ATCC) was about 6%.

It showed effective results at E:T ratio of 1:3 in the co-culture condition with NIH/3T3 (ATCC) by M2 BMDM treated with MPEI/pCAR in the co-culture condition with NIH/3T3 (ATCC), as compared to the cell ratio of NIH/3T3 (ATCC) by M2 BMDM treated with MPEI/pGFP in co-culture condition with NIH/3T3 (ATCC).

### Example 6

After seeding the RAW 264.7 (ATCC) cell line into a 6-well plate (SPL) at a concentration of 5×10⁵ cells/well, 16 hours later, the lentivirus containing pCDH_CAR plasmid was treated for 24 hours, followed by isolating only RAW 264.7 cells expressing CAR through FACS AriaII (BD Biosciences).

Thereafter, the isolated RAW 264.7 cell line expressing CAR was stained with CELLTRACE™ Cell Proliferation Kit (Thermo-Fisher Scientific).

The isolated RAW 264.7 cells as effector cells, and Neuro2A expressing ALK stained with CELLTRACE™ Red Cell Proliferation Kit (Thermo-Fisher Scientific) as target cells were subjected to co-culture at ratios of Effector cells:Target cells (E:T ratio) of 1:1, 1:3 and 1:5, respectively, 48 hours later, followed by analysis through flow cytometry using a FACSCalibur (BD Biosciences).

The isolated RAW 264.7 cells as effector cells, and NIH/3T3 not expressing ALK stained with CELLTRACE™ Red Cell Proliferation Kit (Thermo-Fisher Scientific) as target cells were subjected to co-culture at E:T ratio of 1:1, 1:3 and 1:5, respectively, 48 hours later, followed by analysis through flow cytometry using a FACSCalibur (BD Biosciences).

A decrease in the cell ratio of Neuro2A (ATCC) was observed in the co-culture condition with the CAR-expressing RAW264.7 cell line and the ALK-expressing Neuro2a, the number of Neuro2A (ATCC) was decreased by about 81% at E:T ratio of 1:3 in the co-culture condition with Neuro2A (ATCC), and effective results were demonstrated in terms of the decrease range of the cell ratio of NIH/3T3 (ATCC) by the RAW264.7 cell line expressing CAR in the co-culture condition with NIH/3T3 (ATCC) as compared to the cell ratio of NIH/3T3 (ATCC) by the RAW264.7 cell line not expressing CAR in the co-culture condition with NIH/3T3 (ATCC).

A decrease in the cell ratio of Neuro2A (ATCC) was observed in the co-culture condition with the CAR-expressing RAW264.7 cell line and the ALK-expressing Neuro2a, the number of Neuro2A (ATCC) was decreased by about 57% at E:T ratio of 1:3 in the co-culture condition with Neuro2A (ATCC), and ineffective results were demonstrated in terms of the decrease range of the cell ratio of NIH/3T3 (ATCC) by the RAW264.7 cell line expressing CAR in the co-culture condition with NIH/3T3 (ATCC) as compared to the cell ratio of NIH/3T3 (ATCC) by the RAW264.7 cell line not expressing CAR in the co-culture condition with NIH/3T3 (ATCC).

Therefore, it can be seen that the ratio of Neuro2A (ATCC) cells is decreased as the ratio of BMDM as an effector cell is increased in the E:T ratio. Accordingly, it was confirmed that the decrease range in the ratio of Neuro2a (ATCC) cells was specifically shown at E:T ratio of 1:3.

A decrease in the cell ratio of Neuro2A (ATCC) was observed in the co-culture condition with the CAR-expressing RAW264.7 cell line and the ALK-expressing Neuro2a. Further, it was confirmed that the number of cells with phagocytosis at E:T ratio of 1:3 in the co-culture condition with Neuro2A (ATCC) was about 8%.

It showed effective results at E:T ratio of 1:3 in the co-culture condition with NIH/3T3 (ATCC) by RAW264.7 cell line expressing CAR in the co-culture condition with NIH/3T3 (ATCC), as compared to the cell ratio of NIH/3T3 (ATCC) by RAW264.7 cell line not expressing CAR.

### Example 7

In order to investigate which cell the produced nanoparticles are introduced into the most in vivo, polyethyleneimine (MPEI) as a cationic polymer with a mannose group, CAR and M1 inducer genes were diluted in 150 mM NaCl, and then mixed with a mixing ratio of nitrogen present in PEI phosphate present in the gene, that is, Nitrogen/Phosphate (N/P) of 6, followed by reacting at room temperature for 30 minutes to complete nanoparticles. At this time, the GFP fluorescent gene was included in the gene.

Six-week-old female A/J mice were purchased from Central Laboratory Animals (Korea) and bred in a kennel with a one-week acclimatization period. After the acclimatization period was completed, the mice were anesthetized with iPran solution, and then 1×10⁶ RFP-expressing cancer cells were injected subcutaneously for cancer modeling. When a size of the cancer reached about 80 mm³, MPEI/pCAR-IFN-γ nanoparticles were directly injected into the cancer tissue.

24 hours after injection, the cancer was separated from the main organs and subjected to fluorescence imaging. Then, after the cancer tissue was made into single cells, immunostaining was performed using specific markers of various immune cells (dendritic cells: CD11c, B cells: CD19, macrophages: CD11b, CD4 T cells: CD4, CD8 T cells: CD8), and the expression of CAR in tissues and cells was investigated using FACS CantoII (BD Biosciences).

Compared to the group injected with only the solution containing nanoparticles (Vehicle), CAR expression was the highest in cancer tissues than other organs. Further, as compared to other immune cells (dendritic cells, B cells and T cells) in cancer tissues, it was confirmed that CAR was most expressed in macrophages.

For immunohistochemical analysis, the cancer tissue was extracted 24 hours after injection of the control group and MPEI/pCAR-IFN-γ, and then fixed in 4% paraformaldehyde solution. Following this, the tissue was frozen with a cryo-tissue embedding agent (OCT compound), and the cancer tissue was sectioned into 10 μm using a cryosectioning machine to prepare tissue slides.

To identify CAR-expressing macrophages, immunohistochemistry was performed using a fluorescent antibody binding to GFP, and RFP-expressing cancer cells and GFP-expressing macrophages were identified through a fluorescence microscope. CAR-expressing macrophages in cancer tissues overlapped with cancer cells or appeared close to the fluorescence, such that CAR-expressing macrophages preying on cancer cells could be observed.

### Example 8

In order to investigate the toxicity of nanoparticles in vivo, 6-week-old female A/J mice were purchased from Central Laboratory Animals (Korea) and bred in a kennel with a one-week acclimatization period. After the acclimatization period was completed, the mice were anesthetized with iPran solution, and then 1×10⁶ cancer cells were injected subcutaneously for cancer modeling. When a size of the cancer reached about 80 mm³, a control group was injected with a solution containing nanoparticles only (Vehicle), while MPEI/pCAR-IFN-γ nanoparticles were directly injected into the cancer tissue.

For several hours (one day before cancer modeling, one day, 11 days, and 18 days after cancer modeling), blood was extracted from mice, and the changes in the liver and kidney-related enzymes [aspartate aminotransferase (AST) and alanine aminotransferase (ALT), creatinine and blood urea nitrogen (BUN)] were investigated using a DRI-CHEM 3500S chemistry analyzer (Fujifilm, Japan).

It was confirmed that the difference between the corresponding enzymes in each plasma of the vehicle-injected mouse and the MPEI/pCAR-IFN-γ-injected mouse was not statistically significant. From these results, it was concluded that MPEI/pCAR-IFN-γ does not have significant toxicity in vivo.

### Example 9

In order to investigate the cancer treatment effects of the injected MPEI/pCAR-IFN-γ, cancer-modeled mice were prepared as in Example 8, and then nanoparticles were directly injected into the cancer to measure changes in cancer size. As a control group, the group injected only with a solution containing nanoparticles (Vehicle), the nanoparticle group containing a gene that is not a CAR-M1 inducer (MPEI/Mock), the nanoparticle group containing only the CAR gene (MPEI/pCAR), and the nanoparticle group containing only M1 inducer (MPEI/pIFN-γ) were established.

Compared with other control groups, it was observed that the group treated with MPEI/pCAR-IFN-γ had the most statistically significant inhibition of cancer growth and also showed an increase in the survival rate of mice.

On the 16th day after cancer modeling from cancer model mice treated with nanoparticles in the same manner as above, cancer tissues were isolated and fixed in 4% paraformaldehyde solution. Then, the tissue was frozen with a cryo-tissue embedding agent (OCT compound), and the cancer tissue was sectioned into 10 μm using a cryosectioning machine to prepare tissue slides. Thereafter, the cancer tissue slides were analyzed through an optical microscope by staining the nuclear membrane of the cells with a hematoxylin solution and the cytoplasm of the cells with an eosin solution, respectively. Cancer cells have a high ratio of cell nucleus to cytoplasm. In the MPEI/pCAR-IFN-γ treatment group, it was observed that the ratio of cytoplasm to nucleus was decreased.

Apoptotic cells were labeled in green through PROMEGA's DEADEND™ Fluorometric TUNEL System protocol and observed through a fluorescence microscope. As a result, the most apoptotic cells were observed in the MPEI/pCAR-IFN-γ treatment group.

### Example 10

After the experiment was conducted in the same manner as in Example 9, various types of samples were obtained from cancer tissues. Then, changes in the number and function of immune cells before and after MPEI/pCAR-IFN-γ treatment were investigated. RNA was extracted from cells in the cancer tissues using Quiazol solution, complementary DNA (cDNA) was made with reverse transcriptase, and real-time polymerase chain reaction (qRT-PCR) was performed using the prepared cDNA. As a result, it was confirmed that, in MPEI/pCAR-IFN-γ-treated group, the expression levels of markers (Granzyme b) related to activity of cytotoxic T cells and M1 marker (Cd80) were significantly higher than in other groups, and the expression level of M2 marker (Vegf) was significantly reduced.

The cancer tissue was separated into single cells, and specific markers of immune cells were labeled using a fluorescent antibody, and changes in the number and function of the immune cells before and after MPEI/pCAR-IFN-γ treatment were investigated through flow cytometry. As a result, the expression level of the M1 marker (CD86) in the MPEI/pCAR-IFN-γ treatment group was significantly higher than that of other groups, while the expression level of the M2 marker (CD163) was significantly reduced.

After separating the cancer tissue and fixing it in a 4% paraformaldehyde solution, the tissue was frozen with a cryo-tissue embedding agent (OCT compound), and the cancer tissue was sectioned into 10 μm using a cryosectioning machine to prepare tissue slides. The prepared cancer tissue slides were labeled with specific markers of immune cells using a fluorescent antibody and analyzed through a fluorescence microscope. It was confirmed that the expression level of M1 marker (iNOS) in the MPEI/pCAR treatment group was significantly higher than that of other groups, while the expression level of the M2 marker (Arginase-1) was significantly reduced.

### Example 11

After the experiment was conducted as in Example 9, a cancer tissue was obtained, separated into single cells, specific markers of immune cells were labeled using a fluorescent antibody, and changes in the number and function of cytotoxic T cell-regulatory T cells before and after MPEI/pCAR-IFN-γ treatment were investigated through flow cytometry. In order to identify the functional change of cytotoxic T cells, first, the CD3 and CD8 markers expressed on the cell membrane were stained, and then the Granzyme B marker was subjected to intracellular staining. Further, as a result of analyzing the difference in the amount of cells expressing all of CD3, CD8 and Granzyme B between groups through flow cytometry, it was confirmed that the number of activated cytotoxic T cells attacking cancer in the group injected directly with MPEI/pCAR-IFN-γ was increased as compared to other groups. In particular, in the group including pCAR (MPEI/pCAR and MPEI/pCAR-IFN-γ), the substance (Granzyme b) related to the activated function of cytotoxic T cells was increased, therefore, it could be found that CAR expression-dependent macrophage phagocytosis and the presentation of cancer antigen had influence on the activation of cytotoxic T cells.

In order to investigate the quantitative change of regulatory T cells, CD3, CD4 and CD25 markers expressed on the cell membrane were first stained, and then, Foxp3 marker was subjected to intracellular staining. Further, as a result of analyzing the difference in the amount of cells expressing all of CD3, CD4, CD25 and Foxp3 between groups through flow cytometry, it was confirmed that the amount of Treg interfering with anticancer immunity was the most greatly reduced in the group treated with MPEI/pCAR-IFN-γ.

It was confirmed that the most statistically significant anticancer immunity was activated when pCAR and pIFN-γ were delivered together, based on the highest ratio of CD8⁺ T cells/Treg, which is an indicator of immune activation.

After separating the cancer tissue and fixing it in a 4% paraformaldehyde solution, the tissue was frozen with a cryo-tissue embedding agent (OCT compound), and the cancer tissue was sectioned into 10 μm using a cryosectioning machine to prepare tissue slides. The prepared cancer tissue slides were labeled with TGF-β using a fluorescent antibody and analyzed through a fluorescence microscope. As a result of the analysis, TGF-β⁺ area was reduced when treated with the groups containing pIFN-γ (MPEI/pIFN-γ and MPEI/pCAR-IFN-γ). The reason of this result may be considered because the number of TGF-β-secreting M2 macrophages was decreased due to pIFN-γ transduction. Since TGF-β is involved in Treg differentiation, it could be seen that the amount of Treg might be reduced due to the decrease of TGF-β.

As a result of investigating the change in inflammation-related factors by enzyme-linked immunoassay (ELISA) after isolating cancer tissues and extracting only proteins, it was confirmed that an amount of inflammatory factors (TNF-α and IFN-γ) involved in the anticancer function in the cancer tissue was increased, while an amount of factors (IL-4 and IL-10) involved in inhibiting the anticancer function was reduced. From the above results, it can be concluded that the nanoparticles prepared in the present invention are effective in changing the immune environment in the cancer tissues to be cancer-unfriendly and inhibiting cancer growth.

### Example 12

In order to investigate which cell the produced nanoparticles are introduced into the most in vivo, when the produced nanoparticles are injected intraperitoneally instead of by direct cancer injection, nanoparticles were prepared as in Example 7 above. After performing cancer modeling in mice, MPEI/pCAR-IFN-γ nanoparticles were injected intraperitoneally on day 4.

24 hours after injection, the cancer was separated from the main organs and subjected to fluorescence imaging. Then, after the cancer tissue was made into single cells, immunostaining was performed using specific markers of various cells (dendritic cells: CD11c, B cells: CD19, macrophages: CD11b, CD4 T cells: CD4, CD8 T cells: CD8), and the expression of CAR in tissues and cells was investigated using FACS CantoII (BD Biosciences).

Compared to the group injected with only the solution containing nanoparticles (Vehicle), CAR expression was the highest in cancer tissues than other organs. Further, as compared to other immune cells (dendritic cells, B cells and T cells) in cancer tissues, it was confirmed that CAR was most expressed in phagocytes.

For immunohistochemical analysis, the cancer tissue was extracted 24 hours after injection of the control group and MPEI/pCAR-IFN-γ and then fixed in 4% paraformaldehyde solution. Further, the tissues were frozen with a cryo-tissue embedding agent (OCT compound), and the cancer tissue was sectioned into 10 μm using a cryosectioning machine to prepare tissue slides.

To identify CAR-expressing macrophages, immunohistochemistry was performed using a fluorescent antibody binding to GFP, and RFP-expressing cancer cells and GFP-expressing macrophages were identified through a fluorescence microscope. CAR-expressing macrophages in cancer tissues overlapped with cancer cells or appeared close to the fluorescence, therefore, CAR-expressing macrophages preying on cancer cells could be observed.

### Example 13

In order to investigate the toxicity of nanoparticles in vivo, 6-week-old female A/J mice were purchased from Central Laboratory Animals (Korea) and bred in a kennel with a one-week acclimatization period. After the acclimatization period was completed, the mice were anesthetized with iPran solution, and then 1×10⁶ cancer cells were injected subcutaneously for cancer modeling. When a size of the cancer reached about 80 mm³, a control group was injected with only a solution containing nanoparticles (Vehicle) while MPEI/pCAR-IFN-γ nanoparticles were injected intraperitoneally to an experimental group.

For several hours (one day before cancer modeling, one day, 11 days and 18 days after cancer modeling), blood was extracted from mice and the changes in the liver and kidney-related enzymes in plasma [aspartate aminotransferase (AST) and alanine aminotransferase (ALT), creatinine and blood urea nitrogen (BUN)] were investigated using a DRI-CHEM 3500S chemistry analyzer (Fujifilm, Japan).

It was confirmed that the difference between the corresponding enzymes in each plasma of the vehicle-injected mouse and the MPEI/pCAR-IFN-γ-injected mouse was not statistically significant. From this, it was concluded that MPEI/pCAR-IFN-γ does not have significant toxicity in the body.

### Example 14

DOPE (diphosphatidylethanolamine), DOTAP (1,2-dioleoyl-3-trimethylammonium propane), cholesterol and PE-PEG (1,2-distearoyl-sn-glycero-3-phosphoethanolamine-N-[methoxy(polyethylene glycol)) were mixed together (Molar ratio of 4 types of components 1:1:0.1:0.1). After thin film hydration, the mixture was passed through a membrane having pore sizes of 1000 nm and 200 nm (extrusion), thereby producing liposomes.

The liposomes and CAR-IFN-γ-GFP pDNA were admixed at a ratio of N/P ratio=Cationic lipid/pDNA=3 to form a lipoplex. At this time, CAR-IFN-γ-GFP pDNA and PiggyBac transposase pDNA were mixed at a ratio of 1:0.4 and then used.

In order to investigate the efficiency of CAR-IFN-γ-GFP pDNA delivery to macrophages in vitro, RAW 264.7 cells as a macrophage cell line were treated with 10% fetal bovine serum-DMEM high medium in a 6-well plate to grow with 30-40% confluency. After replacement with serum free medium, the lipoplex containing CAR-IFN-γ-GFP pDNA was added at a concentration of 5 μg DNA/ml and cultured. After 2 days, GFP-expressing cells were examined by FACS (FIG. 29).

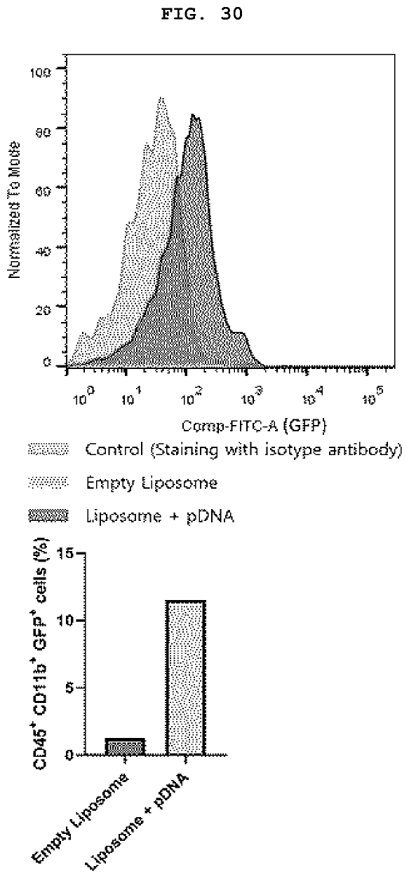

In order to investigate the efficiency of delivering CAR-IFN-γ-GFP pDNA to macrophages in vivo, lipoplex containing 100 μg CAR-IFN-γ-GFP pDNA was injected into the abdominal cavity of C57BL/6 mouse. After extracting cells in the abdominal cavity after 6 hours, APC (CD45, lymphocyte marker)+, PE/Cy7 (CD11b, macrophage marker)+ cells expressing GFP (FITC) among double positive cells were analyzed by FACS (FIG. 30).

### REFERENCE TO AN ELECTRONIC SEQUENCE LISTING

A sequence listing electronically submitted with the present application on Oct. 2, 2023 as an ASCII text file named 20231002_LC0612313_TU_SEQ.TXT, created on Sep. 11, 2023 and having a size of 4,333 bytes, is incorporated herein by reference in its entirety.

1. A composition comprising a complex of a plasmid DNA including a gene which encodes a chimeric antigen receptor (CAR), and a non-viral carrier, and/or a CAR macrophage transformed by the complex.

2. The composition according to claim 1, wherein the chimeric antigen receptor is specifically bound to an antigen selected from the group consisting of anaplastic lymphoma kinase (ALK), CD19, HER2, CD22, CD30, CD73, CD123, FLT3, B-cell maturation antigen, PD-1 MUC16, MSLN, gp100, MART-1, Melna-A, MAGE-A3, MAGE-C2, Mammaglobin-A, proteinsase-3, mucin-1, HPV E6, LMP2, PSMA, GD2, hTERT, PAP, ERG, NA17, GM3, EPhA2, NA17-A, TRP-1, TRP-2, NY-ESO-1, CEA, CA 125, AFP, Survivin, AH1, ras, G17DT, MUC1, E75, p53, PSA, HCG, PRAME, WT1, URLC10, VEGFR1, VEGFR2 and E7.

3. The composition according to claim 1, wherein the plasmid DNA further includes a gene which encodes IFN-γ.

4. The composition according to claim 1, wherein the non-viral carrier is a cationic molecule.

5. The composition according to claim 1, wherein the complex has a ratio (N/P) of nitrogen (N) of the non-viral carrier cationic molecule to phosphorus (P) of the plasmid DNA in a range of 4 to 30.

6. The composition according to claim 1, wherein the complex further includes a transposase plasmid.

7. The composition according to claim 1, wherein the complex is prepared by mixing the plasmid DNA, the non-viral carrier and a transposase plasmid.

8. The composition according to claim 4, wherein the cationic molecule is at least one selected from the group consisting of polyethyleneimine, mannosylated polyethyleneimine, mannosylated cholesterol-polyethyleneimine, PEG-polyethyleneimine-cholesterol, cationic lipids, methacrylate-based polymers including poly[(2-dimethylamino)ethyl methacrylate], polycations including chitosan and beta-cyclodextrin, polyamidoamines, dendrimers, degradable poly((3-esters), poly(lactic-co-glycolic acid), mannosylated liposomes, liposomes containing PEG-cholesterol, PEG-cholesterol-containing liposome and cationic ionizable lipid, and PEG-cholesterol-containing liposomes.

9. The composition according to claim 1, wherein the plasmid DNA further includes a CD8 hinge region, a CD28 transmembrane domain, a CD28 costimulatory domain, or a CD3 zeta signaling domain.

10. The composition according to claim 1, wherein the chimeric antigen receptor is an scFv which binds to a cancer cell surface antigen, a hinge region, a transmembrane domain, an intracellular domain, a costimulatory domain and a signaling domain.

11. (canceled)

12. A method for producing a chimeric antigen receptor (CAR) macrophage, the method comprising transforming a macrophage with a complex of a plasmid DNA including a gene which encodes a chimeric antigen receptor, and a non-viral carrier.

13. The method according to claim 12, further comprising preparing the complex by mixing the plasmid DNA and the non-viral carrier in a ratio of Equation 1 below:
4≤N/P≤30 [Equation 1]
wherein, N is nitrogen of the non-viral carrier cationic molecule, P is phosphorus of the plasmid DNA, and the ratio is the number of elements.

14. The method according to claim 12, wherein the macrophages are transformed in vivo or in vitro.

15. The method according to claim 12, wherein the chimeric antigen receptor is specifically bound to an antigen selected from the group consisting of anaplastic lymphoma kinase (ALK), CD19, HER2, CD22, CD30, CD73, CD123, FLT3, B-cell maturation antigen, PD-1 MUC16, MSLN, gp100, MART-1, Melna-A, MAGE-A3, MAGE-C2, Mammaglobin-A, proteinsase-3, mucin-1, HPV E6, LMP2, PSMA, GD2, hTERT, PAP, ERG, NA17, GM3, EPhA2, NA17-A, TRP-1, TRP-2, NY-ESO-1, CEA, CA 125, AFP, Survivin, AH1, ras, G17DT, MUC1, E75, p53, PSA, HCG, PRAME, WT1, URLC10, VEGFR1, VEGFR2 and E7.

16. The method according to claim 12, wherein the plasmid DNA further includes a gene which encodes IFN-γ.

17. The method according to claim 12, wherein the non-viral carrier is a cationic molecule.

18. The method according to claim 12, wherein the complex further includes a transposase plasmid.

19. The method according to claim 17, wherein the cationic molecule is at least one selected from the group consisting of polyethyleneimine, mannosylated polyethyleneimine, mannosylated cholesterol-polyethyleneimine, PEG-polyethyleneimine-cholesterol, cationic lipids, methacrylate-based polymers including poly[(2-dimethylaminoe)ethyl methacrylate], polycations including chitosan and beta-cyclodextrin, polyamidoamines, dendrimers, degradable poly((3-esters), poly(lactic-co-glycolic acid), mannosylated liposomes, liposomes containing PEG-cholesterol, PEG-cholesterol-containing liposome and cationic ionizable lipid, and PEG-cholesterol-containing liposomes.

20. The method according to claim 12, wherein the plasmid DNA further includes a CD8 hinge region, a CD28 transmembrane domain, a CD28 costimulatory domain, or a CD3 zeta signaling domain.

21. A method for treating a cancer, the method comprising administering to a subject in need there of a composition comprising a complex of a plasmid DNA including a gene which encodes a chimeric antigen receptor (CAR), and a non-viral carrier, and/or a CAR macrophage transformed by the complex.
